(19) United States
(12) Patent Application Publication
(10) Pub. No.: US 2025/0025042 A1
(43) Pub. Date: Jan. 23, 2025

(54) SYSTEM FOR DETERMINING A RELATIVE PERIPHERAL REFRACTION OF AN EYE OF AN INDIVIDUAL AND OPTICAL DEVICE FOR CAPTURING IMAGES OF THE EYE

(71) Applicant: Essilor International, Charenton-Le-Pont (FR)

(72) Inventors: Martha HERNANDEZ-CASTANEDA, Paris (FR); Gildas MARIN, Paris (FR); Bjorn DROBE, Singapore (SG); Marius PELOUX, Palaiseau (FR); Stéphane BOUTINON, Paris (FR)

(73) Assignee: Essilor International, Charenton-Le-Pont (FR)

(21) Appl. No.: 18/708,895

(22) PCT Filed: Nov. 17, 2022
(86) PCT No.: PCT/EP2022/082200

§ 371 Date: May 9, 2024

(30) Foreign Application Priority Data
Nov. 17, 2021 (EP) 21306595.6

(51) Int. Cl. A61B 3/103; A61B 3/14

(52) U.S. Cl. CPC A61B 3/103; A61B 3/14

(57) ABSTRACT

An optical device for capturing images of an eye of an individual, the optical device having a first measurement channel and a second measurement channel. The first measurement channel generates at least one first lighting beam directed toward the eye and along a first axis and to capture at least one first image of the eye when illuminated by the at least one first lighting beam. The second measurement channel is generates at least one second lighting beam directed toward the eye and along a second axis separated from the first axis by at least 5°, for example at least 10° or at least 20° and to capture at least one second image of the eye when illuminated by the at least one second lighting beam. The first measurement channel and the second measurement channel are synchronized together.

### FIELD

Various aspects of this disclosure generally relate to a device to capture images of an eye of an individual and to a system to determine a relative peripheral refraction of the eye of the individual. The individual can also be called subject, patient or user.

### BACKGROUND

This description is related to peripheral refraction measurement which could be useful to screen an early stage of myopia progression or to customize a lens designed for myopia control. This peripheral refraction measurement also allows the personalization of spectacle lenses for improving peripheral vision, this is especially important for sports spectacle lenses.

To realize this screening or this customization this disclosure will propose to use photorefractive apparatus.

The basic function of a photorefractive apparatus is to collect and analyze ocular responses to light stimuli. Light from an external source enters the eye through the pupil and is focused to create a small illuminated spot on the retina. Some of the light from this retinal spot is returned out of the eye through the pupil after interaction with different layers of the eye. The pattern of light exiting the pupil is determined by the optics of the eye and the optomechanical characteristics of the camera of the photorefractive apparatus. This pattern is dominated by an examinee's refractive error (focusing errors of the eye).

When realising this measure of refraction the light stimuli are sent along a gaze axis of the individual, however, it would be also interesting to realize this measure by sending the light stimuli along an axis non-parallel to the gaze axis. This second type of measurement is called off-axis and allows the determination of peripheral refraction or relative peripheral refraction of the eye.

This relative peripheral refraction of the eye would allow a better determination of the refractive error of the eye, therefore there is a need for solutions to determine this relative peripheral refraction.

Peripheral refraction is an off-axis refraction, while relative peripheral refraction is peripheral refraction-central refraction, i.e. the change in refraction from the fovea to the off-axis point of the retina. By extension, this could also be the difference between two other locations on the retina, as long as one of them is considered as a reference point. This could work for eccentric fixation for example.

### SUMMARY

The following presents a simplified summary in order to provide a basic understanding of various aspects of this disclosure. This summary is not an extensive overview of all contemplated aspects, and is intended to neither identify key or critical elements of all aspects nor delineate the scope of any or all aspects. The sole purpose is to present some concepts of one or more aspects in a simplified form as a prelude to the more detailed description that is presented later.

One aspect of this disclosure is an optical device for capturing images of an eye of an individual. The optical device comprises a first measurement channel and a second measurement channel. The first measurement channel is configured to generate at least one first lighting beam directed toward the eye and along a first axis and to capture at least one first image of the eye when illuminated by the at least one first lighting beam. The second measurement channel is configured to generate at least one second lighting beam directed toward the eye and along a second axis separated from the first axis by at least 5°, for example at least 10° preferably at least 20° and to capture at least one second image of the eye when illuminated by the at least one second lighting beam. The first measurement channel and the second measurement channel are synchronized together.

Another aspect of the disclosure is a system for determining a relative peripheral refraction of an eye of an individual. The system comprises the optical device and a calculation module comprising a memory and a processor arranged to execute the steps of measuring a first photorefraction of the eye based on at least the at least one first image, measuring a second photorefraction of the eye based on at least the at least one second image and determining the relative peripheral refraction based on the first photorefraction and the second photorefraction.

Another aspect of the disclosure is a method to determine a relative peripheral refraction of an eye of an individual. The method comprises the step of capturing, using a first measurement channel of an optical device for capturing images of the eye, at least one first image of the eye, capturing, using a second measurement channel of the optical device, at least one second image of the eye, measuring a first photorefraction of the eye based on at least the at least one first image, measuring a second photorefraction of the eye based on at least the at least one second image, determining the relative peripheral refraction based on the first photorefraction and the second photorefraction. The step of capturing the first image and the step of capturing the second image are synchronized.

In this disclosure an open field system and optical device are proposed. The system and optical device are not very restrictive with the positioning furthermore they have a reasonable cost.

### DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1 represents an embodiment of the system of this divulgation.

FIG. 2 represents an embodiment of the calculation module.

FIG. 3 represents an embodiment of the optical device.

FIG. 4 represents another embodiment of the optical device.

FIGS. 5-a and 5-b show the typical configuration of the localizations of the light sources.

FIG. 6 represents an embodiment in which both measurement channels are located on a single module.

FIG. 7 represents the light beams coming simultaneously from one LED of the first measurement channel and one LED of the second measurement channel.

The FIG. 8 represents the light beams propagating from the retina to the measurement channels.

The FIG. 9 represents an embodiment of the optical device.

### DETAILED DESCRIPTION OF EMBODIMENTS

The detailed description set forth below in connection with the appended drawings is intended as a description of various possible embodiments and is not intended to represent the only embodiments in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

One of the embodiments, represented in FIG. 1, concerns a system 101 for determining a relative peripheral refraction of an eye of an individual.

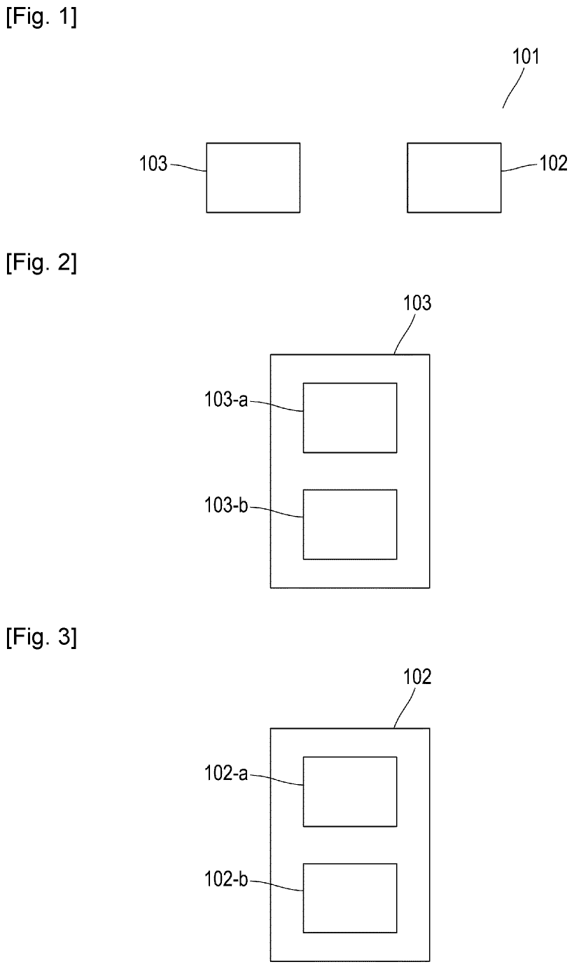

The system 101 comprises an optical device 102 for capturing images of the eye and calculation module 103. As represented in FIG. 2, this calculation module 103 comprises a memory 103-a and a processor 103-b coupled to the memory 103-a.

In an embodiment, the system 101 is a mobile device and the optical device 102 is configured to be removably fastened to a housing of the mobile device and the calculation module 103 is embedded into the mobile device.

In an embodiment, the optical device 102 is an auto-refractometer or an aberrometer and the calculation module being a computer linked to the auto-refractometer or the aberrometer.

Examples of processors 103-b include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure.

The memory 103-a is computer-readable media. By way of example, and not limitation, such computer-readable media may include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

As presented in FIG. 3, the optical device 102 comprises a first measurement channel 102-a and a second measurement channel 102-b. In another embodiment the optical device 102 comprises more than two measurement channels.

The first measurement channel 102-a is configured:
to generate first lightings beam directed toward the eye and along a first axis and
to capture first images of the eye when illuminated by the first lighting beams.

The second measurement channel 102-b is configured:
to generate second lighting beams directed toward the eye and along a second axis separated from the first axis by at least 10° and
to capture second images of the eye when illuminated by the second lighting beams.

The first measurement channel 102-a is used for on-axis refraction and the second measurement channel 102-b is used for off-axis refraction.

The first axis can be a gaze axis of the individual.

The first measurement channel 102-a and the second measurement channel 102-b are synchronized (with wire or wireless) to measure simultaneously the two refractions in the same state of accommodation, to get the accurate refraction, for example spherical equivalent, variation between the two positions.

The memory 103-a is configured to store a computer program comprising instructions which, when the program is executed by the processor 103-b, cause the calculation module 103-b to carry out the steps of
measuring a first photorefraction of the eye based on the first images;
measuring a second photorefraction of the eye based on the second images;
determining the relative peripheral refraction based on the first photorefraction and the second photorefraction.

This system 101 allows the determination of the relative peripheral refraction in a fast and comfortable test, and it is especially adapted for kids. This system 101 can be an open field solution, especially when the system 101 is a mobile device, and is not very restrictive with the positioning.

When the system 101 is an auto-refractometer the system 101 is designed for on-axis and off-axis refraction measurement.

The system 101 allows the management of the accommodation during the on-axis measurement and the off-axis one.

This system also allows the determination of the difference between the refraction of the central part of the retina and the refraction of the peripheral part of the retina, or also between any two other parts of the retina.

The FIG. 4 describes an embodiment of the optical device 102, with the first measurement channel 102-a on-axis to get the standard refraction, this first measurement channel 102-a is aligned with a gazing point (it could also be directly the gazing point). The second measurement channel 102-b is positioned to shape an angle with the first axis, typically at 25° or 30° and approximately at the same distance from the eyes. This angle could be between 5° and 40°. On FIG. 4, the second measurement channel 102-b is shifted horizontally but it could be in another direction (vertically for example). The second measurement channel 102-b could measure by this configuration the off-axis (or peripheral) refraction.

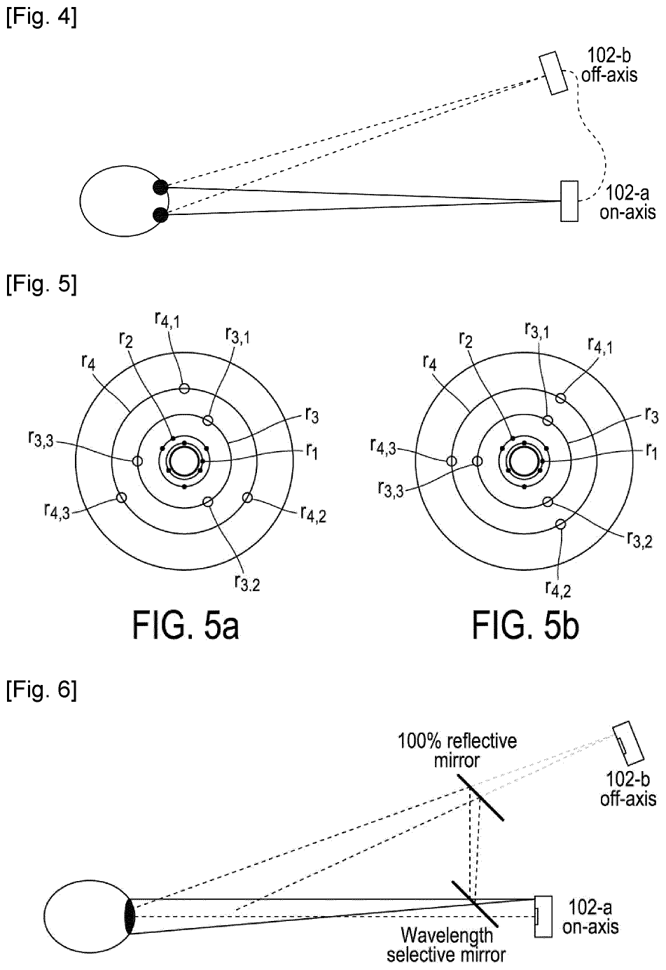

FIGS. 5-a and 5-b show the typical configuration of the localizations of the light sources. The measurement channel comprises a central near-infrared (NIR) camera and 12 light sources positioned at 4 distances from the edge of the camera aperture. These light sources are also positioned to cover different meridians to measure the eye power on different meridians and then to calculate the standard refraction with Spherical parameters, Cylindrical parameter,s Axis or SE, J0, J45 and/or higher order aberrations. The parameters SE, J0 and J45 are described in the article “Power Vectors: An Application of Fourier Analysis to the Description and Statistical Analysis of Refractive Error. Optometry and Vision Science.” of Thibos LN; Wheeler W; Horner, D. published in 1997 in the journal Optom Vis Sci. 1997 June; 74 (6): 367-75

In the FIGS. 5-a and 5-b the light sources are located on four rings r₁ to r₄. The light sources of the same ring are located at the same distance from the border of the aperture of the camera. This distance is also known as eccentricity. In FIGS. 5-a and 5-b, each ring comprises light sources but only the light sources of rings r₃ and r₄ are with references. The ring r₃ comprises three light sources r_{3,1}, r_{3,2} and r_{3,3}. Th ring r₄ comprises three light sources r_{4,1}, r_{4,2} and r^{4,3}. In a nutshell r_{i,j} designates the light source j of the ring i.

Therefore in this embodiment, the first measurement channel 102-a comprises a first camera and the second measurement channel 102-b comprises a second camera. The first camera and/or the second camera can also be configured to determine an intensity distribution of the reflection of the illuminating beam.

In this embodiment, the first measurement channel 102-a comprises a plurality of first light sources for example first LEDs, the second measurement channel 102-b comprises a second plurality of second light sources for example second LEDs. The first measurement channel 102-a comprises between 8 and 14 light sources preferably 12 light sources. The second measurement channel 102-b comprises between 8 and 14 light sources preferably 12 light sources.

The first measurement channel 102-a and the second measurement channel 102-b are synchronized. In the previous embodiment, the device is configured to enlight sequentially one of the first light sources of the first measurement channel 102-a and then one of the second light sources of the second measurement channel 102-b or to enlight sequentially at least two, for example the whole of, the first light sources and then at least two, for example the whole of, the second light sources.

This synchronisation avoids that the accommodation changes during the measurement time. Indeed one of the most interesting parameters of peripheral refraction is the variation of spherical equivalent between off-axis and on-axis; the accuracy of this measurement could be directly impacted by a variation of accommodation. This system 101 allows a simultaneous measurement (or very quick) to avoid this error. Moreover, as we are looking at the difference between both measurements, it is not mandatory to control accommodation (or know at what distance the individual is looking) for the measurement, as for normal refractions, even if the accommodation can induce small changes in peripheral refraction.

The full measurement with 12 light sources typically lasts 250 ms (˜20 ms to capture an image using one of the light sources), so the full sequence (on-axis and off-axis) will take 500 ms. Micro-fluctuations of accommodation (usually 0.1 to 4 Hz and amplitude of less than 1 diopter) could really affect the process. Using the embodiments of this divulgation we can optimize the synchronization between the two measurement channels to mitigate the effect of the accommodation's micro-fluctuations.

In an embodiment, we alternate quickly the 2 measurements. The system 101 is configured to allow the communication and the synchronization of the two measurement channels 102-a and 102-b. The system 101 is configured to quickly alternate the measurement of the first measurement channel 102-a and the second measurement channel 102-b. For example: LED 1 of the first measurement channel 102-a, LED 1 of the second measurement channel 102-b, LED 2 of the first measurement channel 102-a, LED 2 of the second measurement channel 102-b, etc. This alternate of the measurement allows the reduction of the timing between off-axis (with the second measurement channel 102-b) and on axis (with the first measurement channel 102-a) at less than 40 ms for the same measurement. By same measurement, we mean the measurement is from the same light sources relative to the camera.

In an embodiment, we realize the following steps:
full measurement with the first measurement channel 102-a. By full measurement we mean a measurement using successively the whole first light sources of the first measurement channel 102-a,
selection of the best ring rᵢ based on the refraction,
alternate fast measurement: measurement using a first LED r_{i,1} of the ring rᵢ of the first measurement channel 102-a LED followed by measurement using a first LED r_{i,1} of the ring rᵢ of the second measurement channel 102-b and followed by measurement using a second LED r_{i,2} of the ring rᵢ of first measurement channel 102-a LED . . .
Or measurements using successively all the LEDs r_{i,1}, r_{i,2}, r_{i,3} of the ring rᵢ of the first measurement channel 102-a LED followed by measurements using successively all the LEDs r_{i,1}, r_{i,2}, r_{i,3} of the ring rᵢ of the second measurement channel 102-b LED.

In an embodiment, we realize the following steps:
Measurement off-axis with accommodation control (on-axis)
Full measurement on-axis
Selection of the best LED of module 1 to monitor the accommodation
Measurement off-axis while measuring accommodation on-axis

In an embodiment, the first lighting beam of the first measurement channel 102-a has a first optical wavelength and the second lighting beam of the second measurement channel 102-b has a second optical wavelength different from the first optical wavelength.

For example, the first optical wavelength is comprised between 800 nm et 899 nm and the second optical wavelength is comprised between 901 nm and 1000 nm. Otherwise one of the optical wavelengths can be a red light and the other can be a green light.

More precisely, the first measurement channel 102-a can comprise LEDs emitting the first light beam at 860 nm and the camera of the first measurement channel 102-a can comprise a lens filtering the light beam with limited bandwidth (+30 nm). And the second measurement channel 102-b (for off-axis measurement) can comprise LEDs with a second optical wavelength superior to 900 nm. Therefore the second measurement channel 102-b does not interfere with the first measurement channel 102-a, and then it is possible to realize the measurement for both measurement channels simultaneously.

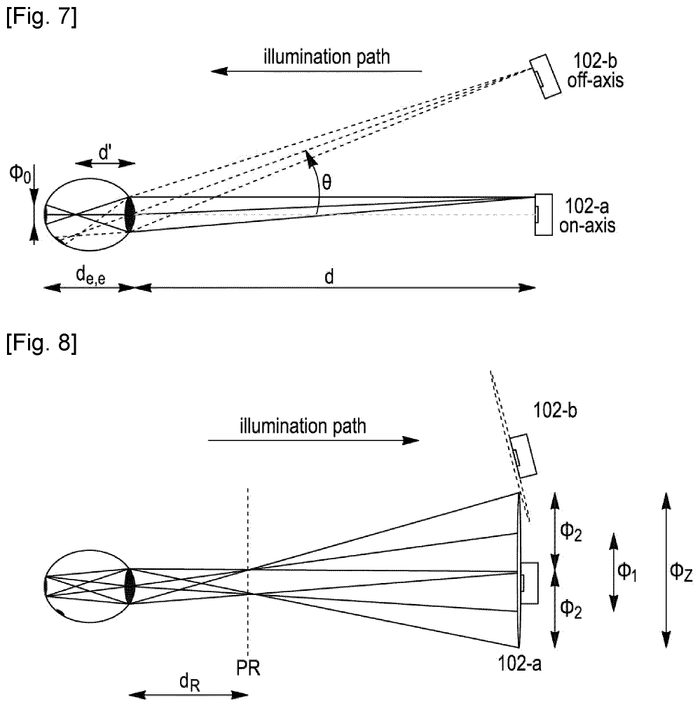

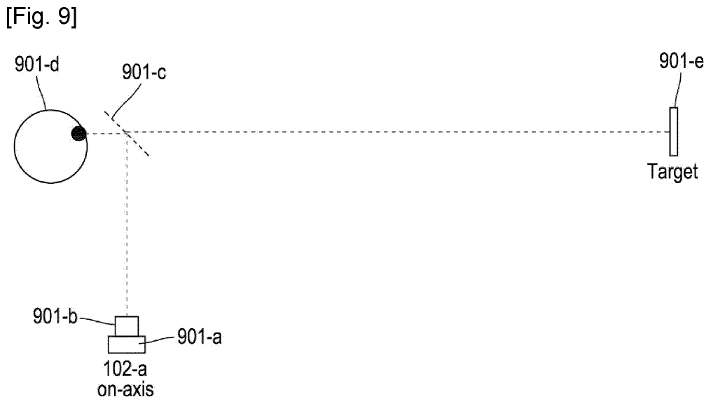

Advantageously, the system 101 corrects the shift of power due to the wavelength. This shift is already corrected at 860 nm to calculate the refraction in the visible (˜-0.9D Φ860 nm). The article “The chromatic eye: a new reduced-eye model of ocular chromatic aberration in humans.” of Larry N Thibos, Ming Ye, Xiaoxiao Zhang, and Arthur Bradley published in AppliedOptics 31, 19 (1992), 3594-3600.), describes, especially in FIG. 6, the correction to get refraction Φ550 nm (central visible) from the measured wavelength.

In an embodiment, described in FIG. 6, both measurement channels are a single module whose sensor has at least two acquisition channels corresponding to two different wavelengths. The optical device 102 comprises one hot mirror 601 that is transmissive for a first optical wavelength, reflective for the second optical wavelength. The optical device 102 also comprises one regular mirror that is reflective for the first optical wavelength and the second optical wavelength. These two mirrors allow the generation and reception of the first lighting beam and the second lighting beam. This embodiment has the advantage of simplifying the synchronization.

In an embodiment, the two measurement channels simultaneously generate light beams having the same wavelength. The two measurement channels are synchronized and if the axis of direction of the first light beam and the axis of direction of the second light beam are separated by more than 20°, they are not interfering with each other.

FIG. 7 represents the light beams coming simultaneously from one LED of the first measurement channel 102-a and one LED of the second measurement channel 102-b. The light propagates here in the “illumination path”. The measurement distance (distance between the eye and the optical device 102) is denoted d, the angle between the two measurement channels is 0. With a very simple eye model, we consider an eye of length d_{e,e} whose crystalline lens is too much convergent (myopic eye). The eye here is made of air between the lens and the retina. The light coming from one LED is focused at a distance d′ behind the crystalline lens and forms a spot of diameter Φ₀ on the retina. The aim is to check that both spots associated to each measurement channel do not overlap.

Let S be the spherical needs of the individual to see correctly. We understand that Φ₀ increases with |S| and with the eye pupil diameter Φₚ. We can show that based on geometrical, paraxial optics, we have:

$${Φ}_{o} = {Φ}_{p} \times \frac{\frac{1}{{P}_{e , e}} - \frac{1}{{P}_{e} - \frac{1}{d}}}{\frac{1}{{P}_{e} - \frac{1}{d}}}$$

P_{e,e} corresponds to the dioptric power of an emmetropic eye, and Pₑ is the dioptric power of the myopic eye, with Pₑ=P_{e,e}−S.

With S=−10δ, Φₚ=8 mm, P_{e,e}=60δ, d=1 m, we get Φ₀=1.2 mm. We compare this value with d_{e,e}×tanθ=6.1 mm for θ=20°. Hence we see that even in this extreme case Φ₀<d_{e,e}×tanθ and then the two spots do not overlap.

The FIG. 8 presents the light beams propagating from the retina to the measurement channels. The light propagates here in the “observation path”. For simplicity's sake only light beams coming from the first spot on the retina are considered. These light beams form a bundle of diameter Φ_{z} in the plane of the module, centered on the LED. The brightness pattern observed by the module on the individual's eye is a result of the intersection of this bundle by the camera aperture. If the rays coming from the first measurement channel 102-a enter the second measurement channel 102-b, we can expect the brightness pattern of the individual's eye to be affected, and hence the photorefraction measurement results.

Therefore, the aim is now to check if Φ_{z}/2 is inferior to d×tanθ. We consider rays from the first measurement channel 102-a and potentially reaching the second measurement channel 102-b, but we could invert the measurement channels in the reasoning. Let Φ₁ be the size of the image of the spot size on the retina crossing a pinhole in the center of the pupil of the eye and reaching the module plane, and Φ₂ be the size of the image in the module plane of a point on the retina. It is reasonable to consider that Φ_{z}=Φ₁+Φ₂. Considering the punctum remotum (PR) at a distance d_{R}=−1/S of the pupil, we can show that:

$${Φ}_{1} = {Φ}_{0} {dP}_{e , e} \\ {Φ}_{2} = - {Φ}_{p} S ( d + \frac{1}{S} )$$

We consider that Φ₁=Φ₂, S=−10δ, Φₚ=8 mm, P_{e,e}=60δ, d=1 m, we get Φ_{z}=144 mm. We compare this value with d×tanθ=364 mm for θ=20°. Hence we see that even in this extreme case Φ_{z}/2<<d×tanθ and then the light coming from one module will not affect the other module.

The FIG. 9 represents an embodiment of the optical device 102. For sake of simplicity, only the first measurement channel 102-a is represented in this figure. As in the other embodiment the first measurement channel 102-a comprises the first light sources 901-a and the camera 901-b. In this embodiment, the first measurement channel 102-a comprises a hot mirror 901-c. The hot mirror 901-c is a beam splitter that reflects infrared (IR) light and transmits visible light. In this embodiment, the first light sources 901-a emitted infrared light. This light is reflected by the hot mirror 901-c and illuminates the eye 901-d. The picture of the eye 901-d is captured by the camera 901-b, the hot mirror 901-c allowing the visible light to pass. During the measurement the individual is looking at the target 901-e. This is possible because the visible light can pass through the hot mirror 901-c. In this embodiment, the first light sources 901-a and the camera 901-b are located in the same location. In this embodiment, thanks to the hot mirror 901-c, the target 901-e can be located at different distances from the eye and this allows a better control of the accommodation of the eye.

This embodiment has two main advantages: the measurement channels are more compact and it allows a far point of gaze reducing instrumental accommodation. This can be interesting if a very precise measurement is required or if there is a need to measure peripheral refraction in far and near vision. With this embodiment, it is easy to change the distance of the fixation target.

1. An optical device for capturing images of an eye of an individual, the optical device comprising;
a first measurement channel; and
a second measurement channel,
wherein the first measurement channel is implemented by a first device configured to:
generate at least one first lighting beam directed toward the eye and along a first axis, and
capture at least one first image of the eye when illuminated by the at least one first lighting beam,
wherein the second measurement channel is implemented by a second device configured to:
generate at least one second lighting beam directed toward the eye and along a second axis separated from the first axis by at least 5°, and
capture at least one second image of the eye when illuminated by the at least one second lighting beam, and
wherein the first measurement channel and the second measurement channel are synchronized together.

2. The optical device according to claim 1, wherein the first device further includes a first camera and the second device includes a second camera.

3. The optical device according to claim 2, wherein the first camera and/or the second camera is further configured to determine an intensity distribution of reflection of an illuminating beam.

4. The optical device according to claim 1, wherein the first device further includes a first plurality of first light sources and
wherein the second device further includes a second plurality of second light sources.

5. The optical device according to claim 4, wherein the optical device is configured to enlight sequentially one of the first light sources and then one of the second light sources or to enlight sequentially at least two of the first light sources and then at least two of the second light sources.

6. The optical device according to claim 1, wherein the first measurement channel and the second measurement channel are located on a same single module, and
wherein the optical device further comprises a mirror, the mirror and the same single module being configured to generate the first lighting beam and the second lighting beam.

7. The optical device according to claim 6, further comprising a hot mirror configured to reflect the first lighting beam and to allow the second lighting beam to pass.

8. The optical device according to claim 1, wherein the first lighting beam having a first optical wavelength and the second lighting beam having a second optical wavelength different from the first optical wavelength.

9. The optical device according to claim 8, wherein the first optical wavelength is comprised between 800 nm et 899 nm and the second optical wavelength is comprised between 901 nm and 1000 nm.

10. A system (101) for determining a relative peripheral refraction of an eye of an individual, the system comprising:
the optical device according to claim 1, and
calculation circuitry comprising a memory and a processor configured to:
measure a first photorefraction of the eye based on at least the at least one first image,
measure a second photorefraction of the eye based on at least the at least one second image, and
determine the relative peripheral refraction based on the first photorefraction and the second photorefraction.

11. The system according to claim 10, wherein the system is a mobile device,
the optical device is further configured to be removably fastened to a housing of the mobile device, and
the calculation circuitry is embedded into the mobile device.

12. The system according to claim 10, wherein the optical device is an auto-refractometer or an aberrometer, and
the calculation circuitry is a computer linked to the auto-refractometer or the aberrometer.

13. A method to determine a relative peripheral refraction of an eye of an individual, the method comprising:
capturing, using a first measurement channel of an optical device for capturing images of the eye, at least one first image of the eye;
capturing, using a second measurement channel of the optical device, at least one second image of the eye;
measuring a first photorefraction of the eye based on the at least one first image;
measuring a second photorefraction of the eye based on the at least one second image; and
determining the relative peripheral refraction based on the first photorefraction and the second photorefraction,
wherein the capturing of the first image and the capturing of the second image are synchronized.

14. The method according to claim 13, wherein the capturing of the first image and the capturing of the second image further comprises:
first enlightening the eye using one of a plurality of first light sources of the first measurement channel,
second enlightening the eye using one of a plurality of second light sources of the second measurement channel, once the first enlightening is finished; and
repeating the first enlightening and the second enlightening.

15. The method according to claim 13, wherein the capturing of the first image and the capturing of the second image further comprises:
first enlightening the eye using at least two of a plurality of first light sources of the first measurement channel
second enlightening the eye using at least two of a plurality of second light sources of the second measurement channel, once the first enlightening is finished.

16. The optical device according to claim 1, wherein the second axis is separated from the first axis by at least 10°.

17. The optical device according to claim 1, wherein the second axis is separated from the first axis by at least 20°.

18. The optical device according to claim 4,
wherein the first plurality of first light sources are first LEDs, and
wherein the second plurality of second light sources are second LEDs.

19. The optical device according to claim 2, wherein the first device further includes a first plurality of first light sources and
wherein the second device further includes a second plurality of second light sources.

20. The optical device according to claim 3, wherein the first device further includes a first plurality of first light sources and
wherein the second device further includes a second plurality of second light sources.
